(19) United States
(12) Patent Application Publication
(10) Pub. No.: US 2015/0147745 A1
(43) Pub. Date: May 28, 2015

(54) Method for Operating a Biogas System in a Batch Mode

(71) Applicant: BEKON Holding AG, Unterfoehring (DE)

(72) Inventor: Peter Lutz, Munich (DE)

(21) Appl. No.: 14/599,432

(22) Filed: Jan. 16, 2015

Related U.S. Application Data
(63) Continuation of application No. PCT/EP2013/065041, filed on Jul. 16, 2013.

(30) Foreign Application Priority Data
Jul. 17, 2012 (DE) 102012212505.1
Jul. 16, 2013 (EP) PCT/EP2013/065041

(51) Int. Cl. C12M 1/34; C12M 1/00; C12M 1/107; C12P 5/02; C12Q 3/00

(52) U.S. Cl. CPC C12M 41/34; C12P 5/023; C12Q 3/00; C12M 21/04; C12M 29/24
USPC 435/3; 435/167; 435/286.6

(57) ABSTRACT

A biogas system operates according to the principle of dry fermentation with multiple biogas fermenters that are run in batch mode. One of the biogas fermenters has just been loaded with fresh biomass and the other biogas fermenters are in the state of producing biogas with a higher concentration of methane. The biogas fermenter just loaded with fresh biomass is closed and connected to the other biogas fermenters that are already producing biogas. In addition, there is a return of the biogas mixture from the freshly loaded biogas fermenter to at least one of the other biogas fermenters producing biogas. After expiration of a defined time duration, the return of the biogas mixture from the freshly loaded biogas fermenter is stopped. The defined time duration is determined based on the concentration of methane in the mixture of biogas generated in all of the biogas fermenters.

### CROSS REFERENCE TO RELATED APPLICATION

This application is filed under 35 U.S.C. §111(a) and is based on and hereby claims priority under 35 U.S.C. §120 and §365(c) from International Application No. PCT/EP2013/065041, filed on Jul. 16, 2013, and published as WO 2014/012952 A2 on Jan. 23, 2014, which in turn claims priority from German Application No. 102012212505.1, filed in Germany on Jul. 17, 2012. This application is a continuation-in-part of International Application No. PCT/EP2013/065041, which is a continuation of German Application No. 102012212505.1. International Application No. PCT/EP2013/065041 is pending as of the filing date of this application, and the United States is an elected state in International Application No. PCT/EP2013/065041. This application claims the benefit under 35 U.S.C. §119 from German Application No. 102012212505.1. The disclosure of each of the foregoing documents is incorporated herein by reference.

### TECHNICAL FIELD

The present invention relates to a method for operating a biogas system with a plurality of bioreactors.

### BACKGROUND

It is known from practical experience and for example from European patent application EP0934998A2 that during so-called dry fermentation the material to be fermented is not converted into a liquid phase, as is the case for example in liquid fermentation of biological wastes. Instead, the fermentation substrate introduced into the biogas fermenter is continuously kept moist by withdrawing the percolate at the bottom of the fermenter and then spraying it over the biomass again. In this way, optimal living conditions for the bacteria required for fermentation are achieved. In addition, the temperature can be regulated during the recirculation of the percolate, and the possibility exists of adding additional materials for process optimization. Dry fermentation is necessary, for example, in the case of very dry or highly fibrous fermentation substrates.

It is known, for example, from German patent application DE102008059803A1 that bioreactors or biogas systems operating according to the dry fermentation principle are frequently operated in the so-called batch mode. The term “batch mode” denotes an operating principle in which no additional material is added or removed during the fermentation process. The biomass or fermentation substrate once loaded into the biogas fermenter remains there until the end of the retention time. At the end of the retention time of the fermentation substrate in the fermenter, the respective fermenter chamber is emptied completely and then refilled. The fermented substrate can, for example, be sent for post-fermentation composting. Thus, one drawback of the batch mode is that the operation of the biogas fermenter must always be interrupted at least when the fermenter chamber is to be emptied and refilled with fresh fermentation substrate. In order to continuously generate an adequate quantity of biogas for generating electricity and/or heat, several fermenters must operate in the biogas system on a staggered schedule.

German patent application DE102008059803A1 deals with the above-described problem of stopping the biogas production in individual biogas fermenters in order to empty and to fill that fermenter. As described above, the already fermented biomass must first be removed from the respective biogas fermenter, the new biomass must then be loaded into the biogas fermenter, and the biogas production must be resumed. When a freshly loaded biogas fermenter is started up, the methane content in the biogas produced at first is so low and the contents of carbon dioxide and nitrogen are so high that immediate use of the biogas in a combined heat and power plant is impossible. To master this problem, German patent application DE102008059803A1 suggests partially removing the non-methane constituents of the gas mixture, also called lean gas, and continuing to return the remaining gas mixture with higher methane content back into the biogas fermenter until the methane content is high enough. One drawback of this method, however, is that the gas mixture is only enriched very slowly, and thus the method is not very efficient. Another drawback is that labor-intensive removal of the non-methane constituents is necessary.

German patent application DE102010028707A1 describes a method for process control of percolators and/or fermenters in two-step biogas generation in which a biogas system using dry fermentation with a plurality of fermenters operates in batch mode. In this process, a chronological work flow is suggested in which at the beginning of percolation air is discharged to remove CO₂-rich hydrolysis gas from the fermenter. This CO₂-rich hydrolysis gas, which cannot be used for energy production, can then be used to displace methane-containing hydrolysis gas in a percolator that has not yet reached the end of percolation. This process is regulated by the methane gas content. Although a biogas system constructed according to DE102010028707A1 supplies biogas with a constant and high methane content, the air-discharge operation during the beginning of percolation reduces the efficiency of the device, and the CO₂-rich gas must subsequently be treated in a gas utilization system.

A comparable process, but one in which the CO₂-containing flushing gas is passed through the substrate, is proposed in European patent application EP2251408A1.

German patent application DE102009025329B4 provides a method for gas exchange between two or more bioreactors in which gas from the headspace of one or more bioreactors is introduced into the headspace of one or more other bioreactors. For this purpose, the measured values of H₂S, CO₂, CH₄ and O₂ from all bioreactors are polled, and the exchange between two and more bioreactors is regulated. Pressure and sulfur content are used as the values for determining the time at which a bioreactor is ready for the exchange with one or more other bioreactors. The method specified in DE102009025329B4 increases the efficiency of the biogas system, but it also requires the use of a considerable amount of measurement, monitoring and control engineering.

Therefore, the problem confronting the present invention is that of providing a method for operating a biogas system and a biogas system operated in this manner, in which the efficiency in the batch mode is markedly increased by using a means of the simplest possible design, whereby the continuous generation of usable biogases is achieved.

### SUMMARY

A biogas system that operates with dry fermentation includes multiple biogas fermenters that are run in batch mode. A first biogas fermenter is loaded with fresh biomass. Then the first biogas fermenter is closed. The first biogas fermenter is connected by a gas transfer line to a second biogas fermenter that is already producing biogas with a concentration of methane greater than 40%. Biogas generated in the first biogas fermenter is transferred to the second biogas fermenter through the gas transfer line using a pressure gradient that builds up between the first biogas fermenter and the second biogas fermenter. The transfer of the biogas from the first biogas fermenter to the second biogas fermenter is stopped after a defined time duration has elapsed by closing a valve device along the gas transfer line. The defined time duration is determined based on the concentration of methane in the mixture of biogas generated in both the first biogas fermenter and the second biogas fermenter. Before the first biogas fermenter is connected to the second biogas fermenter and biogas is allowed to flow between the two biogas fermenters, the first biogas fermenter is flushed with an oxygen-free flushing gas or a flushing gas containing as little oxygen as possible. The flushing gas is supplied by a gas processing apparatus connected downstream of the second biogas fermenter.

A biogas system with multiple biogas fermenters is operated in a batch mode by loading a first biogas fermenter with fresh biomass and then closing the first biogas fermenter. The first biogas fermenter has a gas pressure that is initially lower than that of the second biogas fermenter that is already producing biogas with a concentration of methane greater than 40%. The first biogas fermenter is connected by a gas transfer line to the second biogas fermenter. A valve along the gas transfer line is opened to allow biogas with the concentration of methane greater than 40% to flow into the first biogas fermenter because the gas pressure in the first biogas fermenter is initially lower than the gas pressure in the second biogas fermenter.

The value is left open for a defined time duration while the gas pressure in the first biogas fermenter increases to greater than the gas pressure in the second biogas fermenter. The greater gas pressure in the first biogas fermenter causes biogas in the first biogas fermenter to flow into the second biogas fermenter. Biogas is also collected directly from the second biogas fermenter into a biogas collection line. The value along the gas transfer line is closed after the defined time duration has elapsed. The defined time duration is based on the concentration of methane sensed in the biogas collection line, which includes biogas generated in both the first biogas fermenter and the second biogas fermenter.

A biogas system that uses dry fermentation includes multiple biogas fermenters, a biogas collection line and a control device. A first biogas fermenter has a first gas inlet and a first biogas outlet, and a second biogas fermenter has a second gas inlet and a second biogas outlet. The first biogas fermenter that has just been loaded with fresh biomass, while the second biogas fermenter is already producing biogas with a high concentration of methane, such as greater than 40%. A gas transfer line connects the first biogas outlet to the second gas inlet. A valve device is located along the gas transfer line. The control device controls the valve device to allow biogas with a lower methane concentration (lean gas) produced in the first biogas fermenter to be transferred for a defined time duration to the second biogas fermenter through the gas transfer line.

Biogas is removed from the second biogas fermenter through a biogas collection line. The first biogas outlet and the second biogas outlet are connected to the biogas collection line. A methane concentration sensor senses the methane concentration in the gas in the biogas collection line. A data line connects the methane concentration sensor to the control device. The control device controls the valve device based on the methane concentration sensed in the biogas collection line. The first gas inlet on the first biogas fermenter is connected to a source of oxygen-free flushing gas, such as a gas processing apparatus that receives biogas that flows through the biogas collection line. The first biogas fermenter is flushed with the oxygen-free flushing gas before the valve is opened to allow biogas to flow between the first and the second biogas fermenters.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 is a highly schematic top view of a biogas system according to the invention with a plurality of biogas fermenters.

FIG. 2 is a schematic top view of an embodiment of the biogas system according to the invention.

FIG. 3 is a schematic top view of another embodiment of the biogas system according to the invention.

FIG. 4 is a schematic top view of yet another embodiment of the biogas system according to the invention.

### DETAILED DESCRIPTION

Reference will now be made in detail to some embodiments of the invention, examples of which are illustrated in the accompanying drawings.

The invention relates to a method for operating a biogas system with a plurality of biogas fermenters according to the dry fermentation principle in batch mode. At least one of the biogas fermenters has just been loaded with fresh biomass, i.e., within a short time, and one or more of the other biogas fermenters are in a state in which they are producing biogas with a higher methane content, i.e., better for use. The method includes the following steps:
closing the one or more biogas fermenter loaded with fresh biomass,
connecting the one or more biogas fermenters loaded with fresh biomass with one or more of the other biogas-producing biogas fermenters,
transferring the biogas mixture from the one or more freshly loaded biogas fermenters into one or more of the other biogas-producing biogas fermenters, and
ending the transfer of the biogas mixture from the freshly loaded biogas fermenter after a defined time duration has elapsed.

The biogas from the freshly loaded biogas fermenter, so-called lean gas, is returned to a single or simultaneously into several other, currently operating and usable biogas fermenters producing biogas with high methane concentrations. Through the mixing of the lean gas with the biogas of higher methane content from one or more of the other biogas fermenters, the fluctuation of the methane concentration in the mixed biogas from all biogas fermenters and the rate of change of the methane concentration can be reduced in a simple manner. Frequently, the biogas produced in the biogas fermenters is supplied to a gas-powered combined heat and power plant (CHP). Connected before the gas engine is a so-called gas mixer, which mixes the biogas with the air required for combustion and ensures a combustible biogas/air mixture at the different methane concentrations in the biogas. These gas mixers have a certain hysteresis so that in the case of rapid and/or pronounced fluctuations in the methane concentration in the biogas, under certain circumstances a noncombustible biogas/air mixture is fed to the gas engine and the gas engine stalls. The gas transfer according to the present invention reduces the fluctuation of the methane concentration. In addition, the change in the methane concentration in the mixed biogas from all of the biogas fermenters takes place more slowly so that the mixer, despite its hysteresis, can always supply a combustible biogas/air mixture for the gas engine.

The defined time duration depends on the size of the biogas system or on the number of individual biogas fermenters, the size of the individual biogas fermenters and/or the type of biomass to be fermented. The time duration is defined based on empirical information. The time duration is selected such that downstream biogas using systems, e.g., CHP with gas engines, gas processing equipment, etc., can be supplied continuously with usable biogas.

Preferably, the defined time duration during which the transfer of the biogas mixture from the freshly loaded biogas fermenter takes place is input into a control apparatus such that the time duration is parameterizable, i.e., can be varied manually by an operator or automatically by a technical device. For example, the time duration can be altered or set depending on the size of the overall biogas system (the number of biogas fermenters) and depending on the size of the biogas fermenters. By having the control device control the transfer and/or the end of the transfer as a function of the defined time duration, the biogas system can be operated in a particularly flexible and efficient manner.

The biogas system can be operated even more flexibly if the defined time duration can also be modified during ongoing operation of the biogas system by means of additional parameters and/or manually by an operator. For example, the concentration of certain biogas fractions, especially the methane concentration, is measured and on the basis thereof the defined time duration is shortened or lengthened to optimize the operating sequence chronologically and/or qualitatively.

To increase the operational reliability of the biogas system, an oxygen-free flushing gas or a flushing gas containing as little oxygen as possible is introduced into the biogas fermenter after closing the one or more biogas fermenter loaded with fresh biomass. The biogas fermenter is flushed before opening until it is guaranteed that upon opening the biogas fermenter an explosive biogas/air mixture can no longer form as a result of the inflowing air. The risk of an accident is considerably reduced in this way.

The method is particularly simple because the transfer of the biogas mixture from the one or more freshly loaded biogas fermenters takes place through a pressure gradient formed by the simple connection of the biogas fermenter loaded with fresh biomass at ambient pressure with the other biogas fermenters, which are at slightly elevated pressures due to the biogas generated. Thus, no additional auxiliary means, especially no transport means, is necessary.

In an advantageous embodiment of the method for selecting the defined time duration, the methane concentration in the biogas mixture from all biogas fermenters is measured in a biogas collection line. The duration of transfer of the lean gas into one or more already biogas-producing biogas fermenters is selected such that the methane concentration in the biogas collection line does not fall below the minimum value provided by the downstream biogas utilization apparatus. If the methane concentration in the biogas collection line is measured continuously or at intervals, a particularly accurate and efficient operation of the overall biogas system is achieved because all measures for transferring the biogas also occur dependent upon the measured methane concentration or can be guided by it.

In an additional advantageous embodiment, one or more freshly loaded biogas fermenters are not connected only with a single biogas fermenter but also with several of the other biogas fermenters, so that their biogas mixtures are correspondingly transferred to several biogas fermenters. As a result, the efficiency of the method can be further considerably increased again.

It is likewise highly advantageous if the transfer or the introduction of the biogas to be enriched, i.e., of the lean gas, takes place at a point in the other biogas fermenter that is as remote as possible from the point at which the biogas generated in the respective biogas fermenter is withdrawn or removed. As a result, the residence time for enriching the lean gas is extended in a simply designed manner.

A biogas system suitable for carrying out the above described method has a plurality of biogas fermenters that operate according to the dry fermentation principle, whereby each biogas fermenter has one or more biogas outlets and one or more gas inlets. In addition, the biogas system has one or more gas transfer lines with a valve device through which the biogas outlet of a biogas fermenter can be connected with the gas inlet of another one for a defined time duration. This biogas system is advantageously suitable for increasing the methane content of the lean gas of a freshly loaded biogas fermenter in a particularly simple manner and for reducing the hydrogen sulfide content because the biogas is not conducted out of the biogas system, but rather is distributed by the biogas system to one or more other biogas fermenters. Thus, the residence time of the lean gas in the biogas system is prolonged.

A particularly advantageous embodiment of the biogas system according to the invention has a measuring device for determining the methane concentration in a biogas collection line in which the biogas from all biogas fermenters is combined.

It is advantageous for the control device to control and/or regulate the connection of the gas outlet of the one or more freshly loaded biogas fermenters with the gas inlet of one or more of the other biogas fermenters by way of the gas transfer line. In this process, the control/regulation may take place only during the defined time duration and/or also only as a function of one or more parameters of the biogas system. In particular, the methane concentration in the biogas collection may be considered for this purpose.

Ideally the gas inlet for the transferred lean gas and the biogas outlet for the enriched or ready-to-use biogas in the biogas fermenters are separated as far as possible from one another. In particular, these can be arranged at opposite ends of the respective biogas fermenter. In this way, it is possible to enrich the lean gas with methane because in addition to the prolonged residence time, the gas also must cover the longest possible transport route.

The gas inlet can be arranged in the bottom area of the respective biogas fermenter. As a result, the lean gas must pass through the biomass and the residence time of the lean gas in the respective biogas fermenter will be prolonged. Consequently, particularly effective mixing will take place between the lean gas and the biogas that has a high methane concentration generated in the biogas fermenter.

An advantageous embodiment of the invention provides that the gas inlets can be connected to a source of flushing gas with low oxygen content. In this way, a biogas fermenter can be flushed with flushing gas prior to opening and reloading, and the formation of an explosive biogas/air mixture can be definitely prevented.

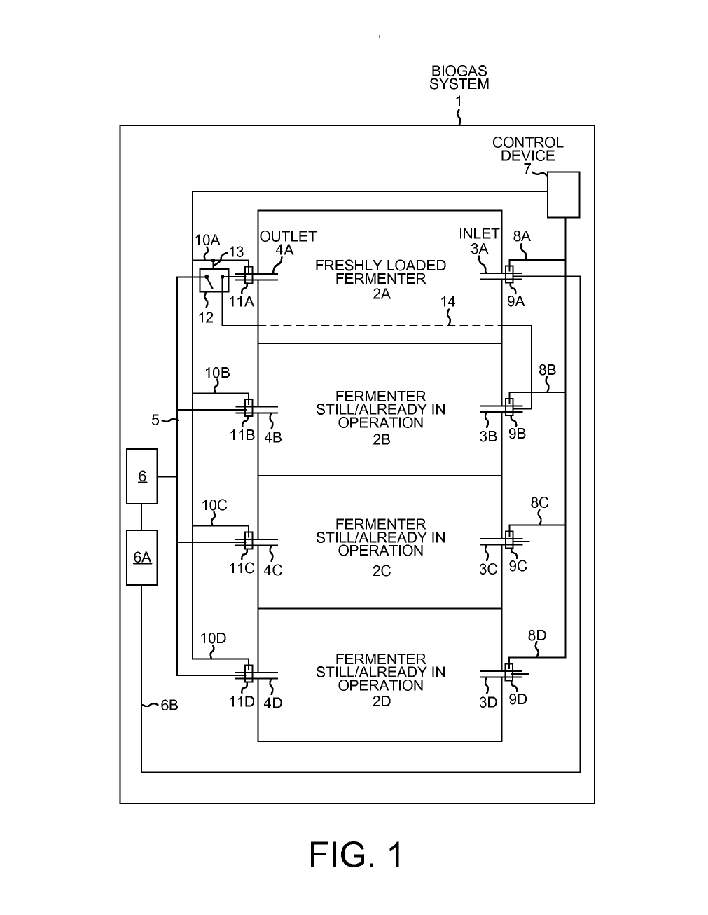

FIG. 1 shows a highly schematic top view of a biogas system 1 according to the invention, which operates according to the dry fermentation principle. The biogas system 1 shown here has several biogas fermenters 2A to 2D for fermenting free-flowing fermentation substrates and biomasses. In this embodiment, exactly four biogas fermenters 2A to 2D are shown, wherein the invention is also applicable to fewer or more biogas fermenters.

The biogas fermenters 2A to 2D are operated in a so-called batch mode, i.e., the biomasses to be fermented remain within each biogas fermenter 2A to 2D during the entire fermentation process, and the biogas fermenters 2A to 2D are operated with staggered timing. Each individual one of the biogas fermenters 2A to 2D has a gas inlet 3A to 3D for the introduction of lean gas from a freshly loaded biogas fermenter or a flushing gas of low oxygen content. The gas inlets 3A to 3D are preferably arranged at the bottom portion or close to the bottom portion of the biogas fermenters 2A to 2D. Each biogas fermenter 2A to 2D has a gas outlet 4A to 4D for removing biogas located at a point that is as far away as possible from the gas inlets 3A to 3D. The location or position of the gas outlets 4A to 4D on the biogas fermenters 2A to 2D is selected such that the transport pathway of the lean gas introduced from the respective gas inlet 3A to 3D to the gas outlet 4A to 4D is as long as possible.

The gas outlets 4A to 4D of the biogas fermenters 2A to 2D are combined over one or more gas lines 5 in a gas collection line 6. Over the gas collection line 6, the biogas produced in the biogas system is delivered to a biogas utilization apparatus 6A, for example a combined heat and power plant 6A. The combined heat and power plant 6A can be connected over an off-gas conduit 6B with the gas inlets 3A to 3D. In this way, off-gas from the CHP 6A can be delivered selectively to individual biogas fermenters as flushing gas.

As shown in FIG. 1, the biogas system 1 also has a control device 7. This is connected over data lines 8A to 8D with valve devices 9A to 9D, respectively, that regulate the respective gas intake at each of the gas inlets 3A to 3D, and is set up to actuate the valve devices 9A to 9D. Furthermore, the control device 7 is connected by way of further data lines 10A to 10D to respective additional valve devices 11A to 11D, which regulate the respective gas discharge at each of the gas outlets 4A to 4D and is set up for corresponding actuation of the valve devices 11A to 11D.

The biogas system 1 also has one or more additional valve devices 12 connected over an additional data line 13 with the control device 7. In addition, one or more gas transfer lines 14 are also provided in the biogas system 1. One end of the gas transfer line 14 can be connected by way of the valve device 12 to one or more of the gas outlets 4A to 4D and the other end with one or more of the gas inlets 3A to 3D of the biogas fermenters 2A to 2D. In other words, one or more of the gas outlets 4A to 4D can be detached from the gas collection line 6 and instead be connected with the gas transfer line 14. In this embodiment, the gas outlet 4A is connected with the gas inlet 3B by way of the gas transfer line 14.

The control device 7 is set up to control and/or regulate the gas inlets 3A to 3D, the gas outlets 4A to 4D and the valve device 12 and to connect and disconnect the gas transfer line 14. In particular, valve device 12 is set up to connect and disconnect the gas transfer line 14 as needed, and instead to connect or disconnect the respective gas outlet 4A to 4D from the gas collection line 6. The demand-guided coupling of the gas transfer is accomplished by the control device 7, which controls or regulates this process in a timed manner and/or as a function of additional parameters. The data for the time control and/or the additional parameters are parameterizable, i.e., programmable.

The operation of the biogas system can take place as follows. The biogas fermenter 2A shown in FIG. 1 is shut down after the complete fermentation of the biomass present therein. After the fermenter 2A is turned off and emptied, it is then in a condition in which it can be loaded or filled with fresh biomass to be fermented. After resumption of operation of the biogas fermenter 2A, it takes some time for the biogas that forms during fermentation to have a methane content or a methane concentration that would allow reasonable and efficient further utilization in a downstream biogas utilization apparatus.

The biogas with a low methane content produced in the freshly loaded biogas fermenter 2A, so-called lean gas, is then transferred to one or more of the other biogas fermenters 2B to 2D. For this purpose, it is possible to determine in the control device 7 whether, at what starting time, and for how long, i.e., for what time duration, a connection and/or transfer of the lean gas from the freshly loaded biogas fermenter 2A into the other biogas fermenter should take place.

When the freshly loaded fermenter 2A is put into operation, the control device 7 activates the gas outlet 4A and the valve device 12 to separate the gas outlet 4A from the gas collection line 6 and instead to connect the gas outlet 4A with the gas transfer line 14. The gas transfer line 14 is thus connected at one end with the gas outlet 4A of the freshly loaded biogas fermenter 2A and at the other end with the gas inlet 3B of the biogas fermenter 2B, which produces comparatively more highly concentrated biogas. In this way, the lean gas is transferred from the biogas fermenter 2A to the biogas fermenter 2B. This transfer takes place for the time duration defined by the control device 7. However, ambient pressure prevails in the freshly loaded biogas fermenter 2A immediately after closing, whereas in the biogas fermenter producing biogas with high methane concentration a slight excess pressure prevails. Thus, in the beginning, this pressure difference causes biogas with high methane concentration to flow into the freshly loaded biogas fermenter 2A. While the biogas fermenters 2A and 2B are connected together in this way, the biogas production in the freshly loaded biogas fermenter 2A begins, and the pressure in the freshly loaded biogas fermenter 2A increases until it is greater than the pressure in the biogas fermenter 2B. Consequently, at first the mixture of lean gas from fermenter 2A and biogas that has entered from fermenter 2B flows back into the biogas fermenter 2B. After this beginning period, only lean gas from fermenter 2A flows into fermenter 2B.

After the predetermined time duration defined by the control device 7 has elapsed, the biogas generated in the freshly loaded biogas fermenter 2A has a high enough methane concentration so that the gas transfer can be ended. If the biogas fermenter 2A is now connected directly with the biogas collection line 6 once again, the biogas quality and the methane concentration in the biogas collection line will remain high enough, i.e., it is above a defined limit value that depends on the downstream biogas utilization apparatus. If the biogas utilization apparatus is a CHP, depending on the gas engine used the methane content must not drop below 40%, for example. The biogas system 1 is now in normal operation during which the biogas from all biogas fermenters 2A to 2D is conducted to the gas collection line 6.

Beginning from the embodiment of FIG. 1, the method according to the invention and the biogas system according to the invention can be modified in numerous ways.

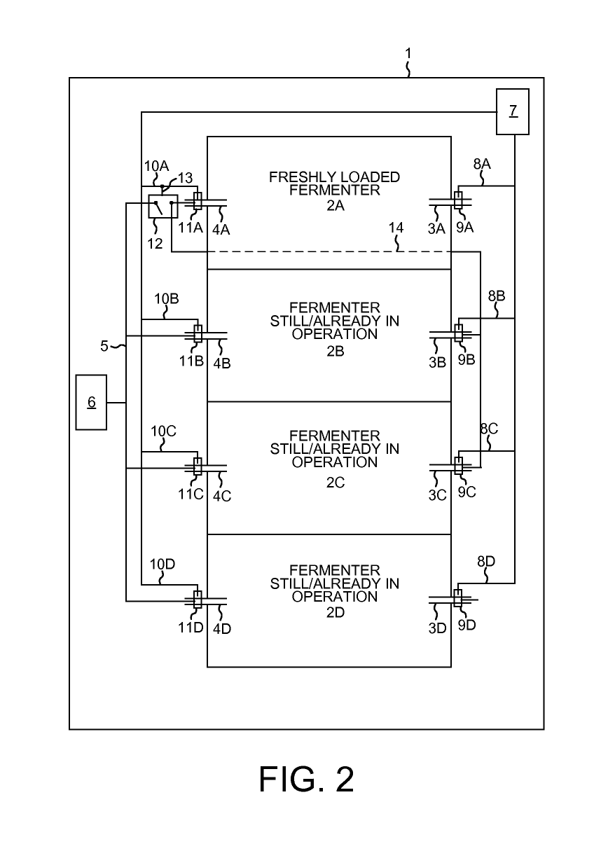

One possible modification is shown in FIG. 2, which shows a schematic top view of the biogas system 1 according to the invention. In this way it is possible for the gas outlet 4A of the biogas fermenter 2A to be connected simultaneously or stepwise with the two gas inlets 3B and 3C, i.e., with more than a single gas inlet of the biogas fermenters 2B to 2D. This means that the lean gas from the biogas fermenter 2A can be distributed over several of the other biogas fermenters 2B to 2D.

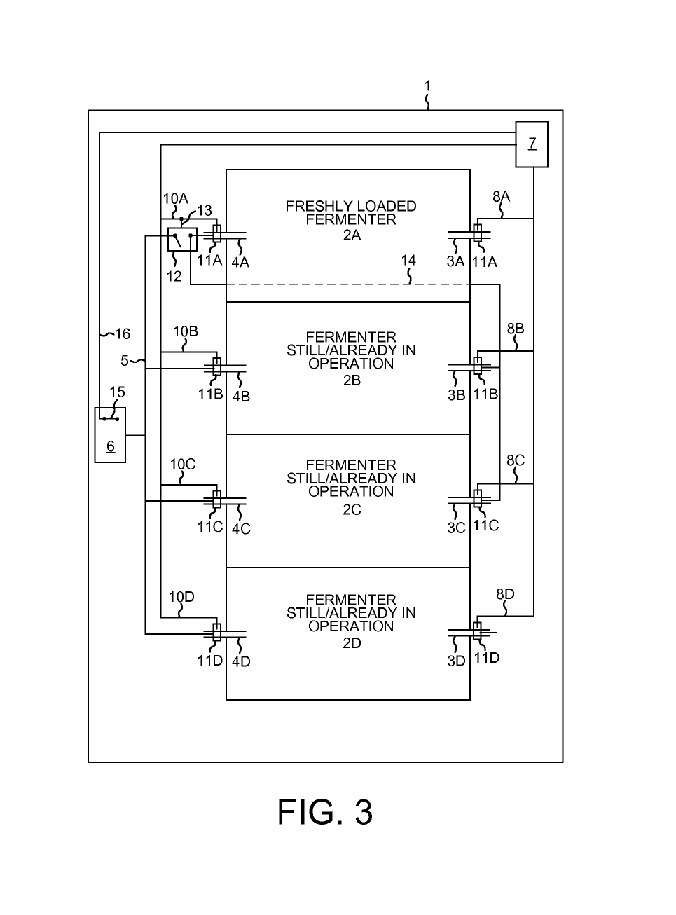

An additional possible configuration of the biogas system 1 is shown in FIG. 3, which likewise shows a schematic top view of the biogas system 1. According to this embodiment, measurement of certain biogas constituents takes place in addition to the above-described time-dependent guidance of the biogas transfer. For this purpose, a measuring device 15 is provided in the biogas collection line 6 for measuring the concentration of the biogas constituents of the biogas mixture, especially the methane concentration. The measuring device 15 is connected to the control device 7 over a data line 16. Thus, the gas transfer from the freshly loaded biogas fermenter 2A can also be controlled and/or regulated as a function of the methane concentration in the biogas collection line 6. In this way, the time duration defined for the gas transfer can be shortened or lengthened depending on the measured biogas constituents. The gas transfer can also be interrupted and resumed.

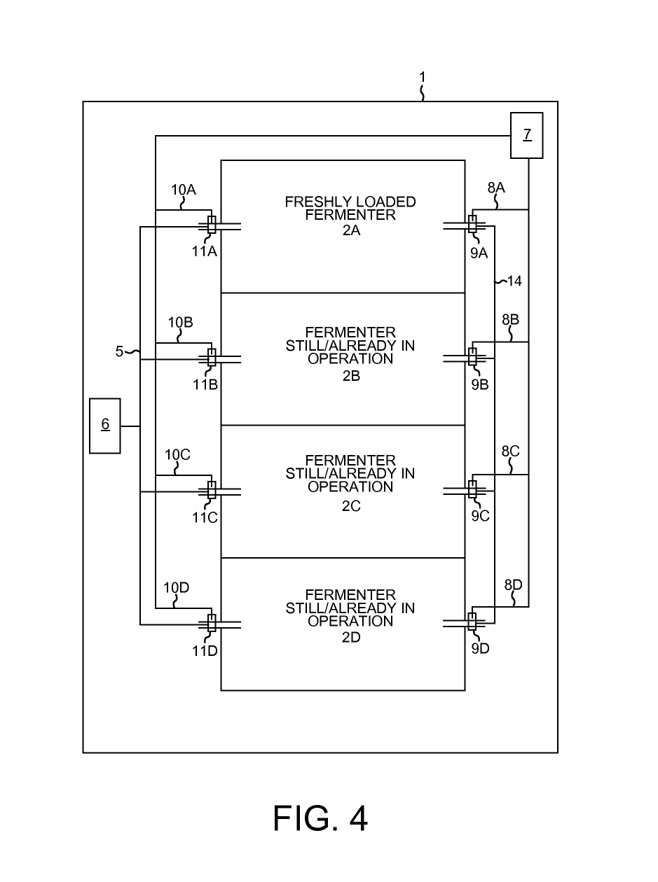

In FIG. 4 an alternative embodiment of the biogas system 1 is presented in a schematic top view. To simplify the design of the biogas system 1 and especially the biogas fermenter 2A to 2D, this embodiment connects the gas transfer line 14 to the gas inlets 3A to 3D. In this way, the line run of the gas transfer line 14 is considerably simplified. In this case, the valve devices 9A to 9D are implemented as 3-way valves. In addition, all valves 10A to 10D can be implemented as 3-way valves so that the number of required components can be further reduced. Altogether this embodiment represents the simplest and most cost-advantageous variant of the biogas system according to the invention 1.

Upon connection of the biogas fermenter 2A that is loaded with fresh biomass to the gas transfer line 14, initially biogas with a high methane concentration flows from the other biogas fermenters 2B to 2D into biogas fermenter 2A. The biogas production (initially lean gas) in the freshly loaded biogas fermenter 2A continues, and the gas pressure in the freshly loaded biogas fermenter 2A rises, so that biogas and lean gas from the freshly loaded biogas fermenter 2A flows into the already biogas-producing biogas fermenters 2B to 2D. The lean gas from biogas fermenter 2A, i.e., biogas of low methane concentration, mixes with biogas of high methane concentration produced in biogas fermenters 2B to 2D. Because of the longer residence time of the lean gas in the biogas system before it reaches the biogas collection line 6, the change in the methane concentration in the biogas collection line 6 proceeds more slowly, so that the gas of a downstream CHP 6A with a gas engine has more time to establish a combustible biogas/air mixture.

In FIG. 4, the freshly loaded biogas fermenter 2A is connected over the gas transfer line 14 with all three of the other biogas fermenters 2B to 2D. Over the control device 7 it is also possible to connect the freshly loaded biogas fermenter 2A to only one or two of the three other biogas fermenters 2B to 2D.

In a biogas system with a larger number of biogas fermenters, it is also conceivable that two or more of the biogas fermenters will be newly filled with fresh biomass simultaneously or with only a slight time difference, and the gas transfer will then take place distributed over the remaining biogas fermenters that were not freshly filled.

### LIST OF REFERENCE NUMERALS

1 biogas system
2A-2D biogas fermenters
3A-3D gas inlets
4A-4D biogas outlets
5 gas line
6 biogas collection line
6A biogas utilization apparatus
7 control device
8A-8D data lines
9A-9D valve devices
10A-10D data lines
11A-11D valve devices
12 valve device
13 data line
14 gas transfer line
15 methane sensor
16 data line

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1-14. (canceled)

15. A method for operating a biogas system that uses dry fermentation, comprising:
closing a first biogas fermenter that has just been loaded with fresh biomass;
connecting the first biogas fermenter to a second biogas fermenter that is already producing biogas with a concentration of methane greater than 40%;
transferring biogas generated by the first biogas fermenter to the second biogas fermenter using a pressure gradient that builds up between the first biogas fermenter and the second biogas fermenter; and
ending the transferring of the biogas from the first biogas fermenter to the second biogas fermenter after a defined time duration has elapsed.

16. The method of claim 15, wherein a value indicative of the defined time duration is stored in a control device, and wherein the ending of the transferring of the biogas is controlled by the control device based on the value.

17. The method of claim 15, further comprising:
flushing the first biogas fermenter with an oxygen-free flushing gas before the connecting of the first biogas fermenter to the second biogas fermenter.

18. The method of claim 17, wherein the flushing gas is supplied by a gas processing apparatus connected downstream of the second biogas fermenter.

19. The method of claim 15, wherein the defined time duration is determined based on the concentration of methane in a mixture of biogas generated in both the first biogas fermenter and the second biogas fermenter.

20. The method of claim 15, further comprising:
removing biogas from the second biogas fermenter, wherein the biogas transferred from the first biogas fermenter to the second biogas fermenter travels the largest possible distance inside the second biogas fermenter before being removed from the second biogas fermenter.

21. A biogas system that uses dry fermentation, comprising:
a first biogas fermenter with a first gas inlet and a first biogas outlet;
a second biogas fermenter with a second gas inlet and a second biogas outlet;
a gas transfer line that connects the first biogas outlet to the second gas inlet; and
a valve device, wherein the valve device is controlled to allow biogas produced in the first biogas fermenter that has just been loaded with fresh biomass to be transferred for a defined time duration to the second biogas fermenter through the gas transfer line while the second biogas fermenter is already producing biogas with a concentration of methane greater than 40%.

22. The biogas system of claim 21, further comprising:
a biogas collection line; and
a methane concentration sensor, wherein the first biogas outlet and the second biogas outlet are connected to the biogas collection line, and wherein the methane concentration sensor senses the methane concentration in the biogas collection line.

23. The biogas system of claim 22, further comprising:
a control device; and
a data line that connects the methane concentration sensor to the control device, wherein the control device controls the valve device based on the methane concentration sensed in the biogas collection line.

24. The biogas system of claim 21, further comprising:
a control device that controls the valve device to allow biogas produced in the first biogas fermenter to be transferred to the second biogas fermenter through the gas transfer line.

25. The biogas system of claim 21, wherein the second gas inlet and the second biogas outlet are located at opposite ends of the second biogas fermenter.

26. The biogas system of claim 21, wherein the second gas inlet is located at the bottom of the second biogas fermenter.

27. The biogas system of claim 21, wherein the first gas inlet is connected to a source of oxygen-free flushing gas.

28. A method comprising:
loading a first biogas fermenter with fresh biomass, wherein dry fermentation begins in the first biogas fermenter;
closing the first biogas fermenter that has just been loaded with fresh biomass, wherein the first biogas fermenter is connected by a gas transfer line to a second biogas fermenter that is already producing biogas with a concentration of methane greater than 40%, wherein the first biogas fermenter has a gas pressure that is initially lower than that of the second biogas fermenter;
opening a valve along the gas transfer line and allowing biogas with the concentration of methane greater than 40% to flow into the first biogas fermenter;
leaving the value open for a defined time duration while the gas pressure in the first biogas fermenter increases to greater than the gas pressure in the second biogas fermenter, wherein the greater gas pressure in the first biogas fermenter causes biogas in the first biogas fermenter to flow into the second biogas fermenter;
collecting biogas directly from the second biogas fermenter into a biogas collection line; and
closing the value after the defined time duration has elapsed, wherein the defined time duration is based on the concentration of methane sensed in the biogas collection line.

29. The method of claim 28, wherein biogas from the first biogas fermenter flows into the second biogas fermenter at the bottom of the second biogas fermenter.

30. The method of claim 28, further comprising:
flushing the first biogas fermenter with an oxygen-free flushing gas before opening the valve.

31. The method of claim 30, wherein the flushing gas is supplied by a gas processing apparatus that receives biogas that flows through the biogas collection line.
